(19) United States
(12) Patent Application Publication
(10) Pub. No.: US 2023/0056368 A1
(43) Pub. Date: Feb. 23, 2023

(54) HEAT- NOT- BURN ARTICLES AND RELATED METHODS AND SYSTEMS

(71) Applicant: 1769474 ALBERTA LTD., Edmonton (CA)

(72) Inventors: Tyler KIBLER, Edmonton (CA); Logan BRAUER, Edmonton (CA); Mark OPPENHEIMER, Edmonton (CA)

(73) Assignee: 1769474 ALBERTA LTD., Edmonton, AB (CA)

(21) Appl. No.: 17/792,950

(22) PCT Filed: Jan. 14, 2021
(86) PCT No.: PCT/CA2021/050032

§ 371 Date: Jul. 14, 2022

Related U.S. Application Data
(60) Provisional application No. 62/960,939, filed on Jan. 14, 2020.

(30) Foreign Application Priority Data
Oct. 29, 2020 (CA) PCT/CA2020/051460

(51) Int. Cl. A61M 11/04; A61M 15/00; A24D 1/20; A24D 1/18; A24D 1/02; A24D 1/00; A24B 15/167; A24D 1/04; A24D 3/17; A24C 5/01

(52) U.S. Cl. CPC A61M 11/041; A61M 15/0001; A24D 1/20; A24D 1/18; A24D 1/02; A24D 1/002; A24B 15/167; A24D 1/04; A24D 3/17; A24C 5/01; A61M 2207/00

(57) ABSTRACT

Heat-not-burn articles are provided. In some embodiments, the heat-not-burn article may comprise: an inner core comprising at least one compressed rod of cannabis-based material; and an outer sheath comprising a sheet of plant-based material, the outer sheath receiving the inner core therein. The heat-not-burn articles may be heated without combustion to generate an inhalable aerosol and/or vapor, thereby reducing or eliminating undesirable byproducts typically associated with smoke. Also provided are related methods and systems for making a heat-not-burn article.

### RELATED APPLICATIONS

The present disclosure claims priority to U.S. Provisional Patent Application No. 62/960,939, filed Jan. 14, 2020, and to PCT Application No. PCT/CA2020/051460, filed Oct. 29, 2020, the entire contents of which are incorporated by reference herein.

### TECHNICAL FIELD

The present disclosure relates articles for consumption of plant material. More particularly, the present disclosure relates to heat-not-burn articles and related methods and systems for making heat-not-burn articles.

### BACKGROUND

Low temperature, non-combustion heating devices may be used to heat plant materials such as tobacco or dried herbs to release volatile components for inhalation by a user. Heating without combustion may prevent the release of toxic byproducts that are typically produced by combustion of plant material.

Some non-combustion heating devices are referred to as “heat-not-burn” devices. One example of a commercially available heat-not-burn device is the IQOS™ system from Phillip Morris International™. Heat-not-burn devices typically comprise a pen-like heater configured to receive a “heat stick” of processed plant material therein. The heat stick may be heated by a heating element of the pen-like heater to aerosolize volatile components of the plant material for inhalation by the user.

Cannabis-based material may be consumed by consumers in a variety of ways including smoking, vaporization, and ingestion. However, there are currently no cannabis-based heat-not-burn articles commercially available. Cannabis-based material may be difficult to form into heat-not-burn articles due to the resinous nature of cannabis plant.

### SUMMARY

In one aspect, there is provided a heat-not-burn article comprising: an inner core comprising at least one compressed rod of cannabis-based material; and an outer sheath comprising a sheet of plant-based material, the outer sheath receiving the inner core therein.

In some embodiments, the cannabis-based material comprises reconstituted cannabis material.

In some embodiments, the reconstituted cannabis material is in the form of a plurality of strips of a reconstituted cannabis sheet.

In some embodiments, the cannabis-based material comprises dried cannabis plant matter.

In some embodiments, the sheet of plant-based material comprises reconstituted cannabis material.

In some embodiments, the sheet of plant-based material comprises cellulosic material.

In some embodiments, the inner core is substantially encased in the outer sheath.

In some embodiments, the sheet of plant-based material is formed into a receptacle and the at least one rod is sealed within the receptacle.

In some embodiments, the cannabis-based material comprises at least one additive.

In some embodiments, the at least one additive comprises at least one of a cannabinoid, a terpene, and a flavonoid.

In some embodiments, the terpene comprises limonene or beta-caryophyllene.

In some embodiments, the heat-not-burn article has a total cannabis content of at least about 90%.

In some embodiments, the at least one rod comprises a first rod and a second rod, the first rod and the second rod being approximately axially aligned in an end-to-end formation.

In some embodiments, the first rod comprises a first chemical composition and the second rod comprises a second chemical composition, the first chemical composition being different from the second chemical composition.

In some embodiments, the inner core further comprises a filter, the filter and the at least one rod being approximately axially aligned in an end-to-end formation.

In some embodiments, each rod of the at least one rod has a first end and a second end and wherein at least one of the first end and the second end defines a recess to receive a heating element of a heat-not-burn device therein.

In another aspect, there is provided a method for making a heat-not-burn article, the method comprising: providing at least one compressed rod comprising cannabis-based material; providing a sheet comprising plant-based material; and wrapping the at least one rod in the sheet.

In some embodiments, providing the at least one rod comprises: providing reconstituted cannabis material; shredding the reconstituted cannabis material into a plurality of strips; and compressing the plurality of strips to form the at least one rod.

In some embodiments, providing the reconstituted cannabis material further comprises infusing the reconstituted cannabis material with at least one additive.

In some embodiments, wrapping the at least one rod in the sheet comprises substantially encasing the at least one rod in the sheet.

In another aspect, there is provided a system for making a heat-not-burn article comprising: a rod-forming device to form a compressed rod of cannabis-based material; and a wrapping device to wrap the rod in a sheet of plant-based material.

In some embodiments, the wrapping device further comprises a sealing mechanism to seal the sheet around the rod.

In some embodiments, the wrapping device comprises a flow-wrapping device.

Other aspects and features of the present disclosure will become apparent, to those ordinarily skilled in the art, upon review of the following description of specific embodiments of the disclosure.

### BRIEF DESCRIPTION OF THE DRAWINGS

Some aspects of the disclosure will now be described in greater detail with reference to the accompanying drawings. In the drawings:

FIG. 1 is a perspective view of an example heat-not-burn article, according to some embodiments;

FIGS. 2A to 2C are perspective views of three other examples of heat-not-burn articles, according to some embodiments;

FIG. 3 is a flowchart of an example method for making a heat-not-burn article, according to some embodiments;

FIG. 4 is a flowchart of another example method showing additional steps for providing a compressed rod of cannabis-based material, according to some embodiments;

FIGS. 5A to 5H are perspective views of various steps of another example method, according to some embodiments;

FIGS. 6A to 6J are perspective views of various steps of another example method, according to some embodiments; and

FIG. 7 is a functional block diagram of an example system for making a heat-not-burn article, according to some embodiments.

### DETAILED DESCRIPTION

Generally, the present disclosure provides a heat-not-burn article. In some embodiments, the heat-not-burn article may comprise: an inner core comprising a compressed rod of cannabis-based material; and an outer sheath comprising a sheet of plant-based material, the outer sheath receiving the inner core therein. Also provided are a related method and system for making a heat-not-burn article.

As used herein and in the appended claims, the singular forms of “a”, “an” and “the” include plural referents unless the context clearly dictates otherwise.

As used herein, “heat-not-burn article” or “smokeless article” may refer to an article that can be heated without combustion to generate an inhalable aerosol and/or vapor. It will be understood that, although the majority of the inhalable substances generated will be aerosol and/or vapor, a small amount of combustion may still be possible and minor amounts of smoke may still be generated.

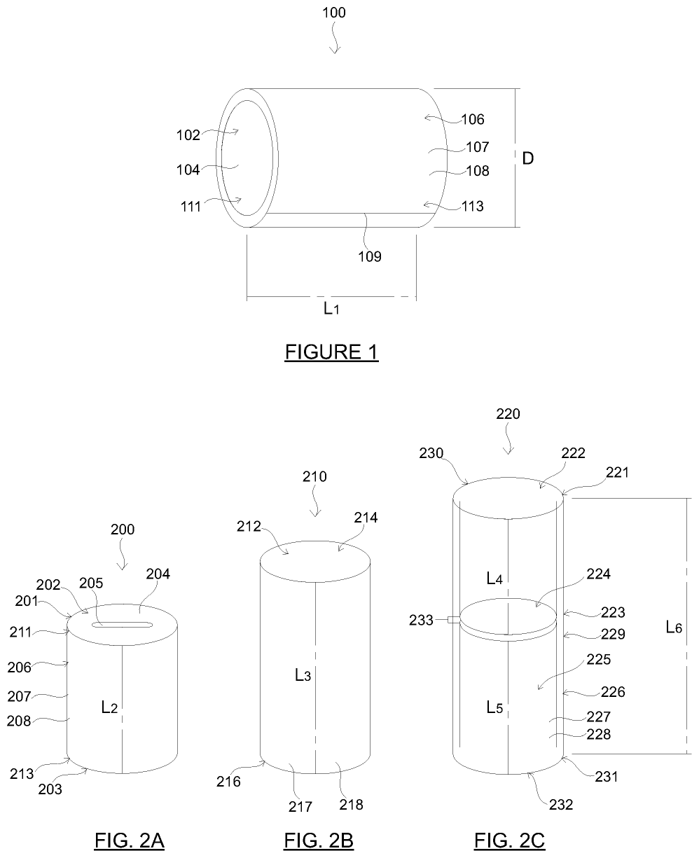

An example heat-not-burn article 100 will be described with reference to FIG. 1. In this example, the heat-not-burn article 100 may be approximately cylindrical in shape. In other embodiments, the heat-not-burn article 100 may be approximately frusto-conical, ovoid, rectangular, or any other suitable shape.

The heat-not-burn article 100 may have a diameter D and a length L₁. The diameter D and the length L₁ may be selected such that the heat-not-burn article 100 may be received into a suitable heat-not-burn device (not shown). In some embodiments, the diameter D may be approximately 8 mm. In other embodiments, the diameter D may be any other suitable diameter. In some embodiments, the length L₁ may be between about 10 mm and about 80 mm. In other embodiments, the length L₁ may be any other suitable length.

The heat-not-burn article 100 may comprise an inner core 102 and an outer sheath 106. The inner core 102 may comprise a compressed rod 104 of cannabis-based material. As used herein, “rod” may refer to an elongate element having a cross-section that is approximately circular, oval, elliptical, square, rectangular, triangular, or any other suitable shape. As used herein, “compressed” or “compressing” may refer to pressing, folding, compacting, or otherwise reducing the size and/or volume of the cannabis-based material of the rod 104. In some embodiments, the cannabis-based material may be compressed relatively firmly to form a relatively solid rod 104. In other embodiments, the cannabis-based material may be compressed relatively lightly to form a relatively loose rod 104. In other embodiments, the inner core 102 may comprise cannabis-based material in any other suitable form.

As used herein, “cannabis-based material” may refer to a material that is substantially obtained or derived from cannabis plant matter but that may also comprise one or more non-cannabis ingredients. As used herein, “cannabis plant” may refer to a plant of the Cannabis genus and “cannabis plant matter” may refer to any substance from one or more cannabis plants. In some embodiments, the cannabis plant may be Cannabis sativa L., Cannabis indica, or Cannabis ruderalis. In some embodiments, the cannabis plant may be a variety or cultivar of cannabis grown for pharmaceutical or recreational applications (e.g. a “drug-type” variety or cultivar). In other embodiments, the cannabis plant may be a variety or cultivar of industrial hemp. In some jurisdictions, industrial hemp must legally contain less than about 0.2% or 0.3% THC (Δ⁹-tetrahydrocannabinol) by dry weight of its leaves and flowers. In other embodiments, the cannabis plant may be any other suitable variety or cultivar.

In some embodiments, the cannabis-based material of the inner core 102 may comprise reconstituted cannabis material. As used herein, “reconstituted cannabis material” may refer to a material produced by processing cannabis plant matter from an original form into a different form.

In some embodiments, the reconstituted cannabis material may be made by the methods described in PCT Application No. PCT/CA2020/051460, filed Oct. 29, 2020, the entire contents of which are incorporated by reference herein. In other embodiments, the reconstituted cannabis material may be made by any other suitable method.

In some embodiments, the reconstituted cannabis material may be produced from cannabis waste matter. As used herein, “cannabis waste matter” may refer to any cannabis plant matter not used in a commercial product, including any unused portion of the cannabis plant, any byproduct from a production process, and/or any other cannabis plant matter that would otherwise be discarded. In some embodiments, the cannabis waste matter may comprise a byproduct produced during the production of cannabis buds, milled flower, or any other commercial cannabis product. In some embodiments, the byproduct may comprise at least one of cannabis leaves, stems, and fine particulate matter (e.g. kief or dust). In other embodiments, the byproduct may comprise any other type of cannabis scraps or post-production material. In other embodiments, the reconstituted cannabis material may be produced from any other suitable cannabis plant matter.

In some embodiments, the reconstituted cannabis material may further comprise one or more excipients. As used herein, the term “excipient” may refer to an additional ingredient that is relatively inert and that is not expected to have biological activity in humans.

In some embodiments, at least one excipient may comprise a humectant. In some embodiments, the humectant may comprise a polyol. In some embodiments, the polyol may comprise a sugar alcohol. Non-limiting examples of sugar alcohols include glycerol (glycerin), erythritol, maltitol, and xylitol.

In some embodiments, at least one excipient may comprise a binding agent. In some embodiments, the binding agent may comprise at least one of a glycol and a polyol. In some embodiments, the glycol may be propylene glycol. In some embodiments, the polyol may be a sugar alcohol. In some embodiments, the sugar alcohol may comprise glycerin, such as vegetable glycerin.

In some embodiments, at least one excipient may comprise a stabilizing agent. In some embodiments, the stabilizing agent may comprise at least one of a glycol, a polyol, and a polysaccharide. In some embodiments, the glycol may be propylene glycol. In some embodiments, the polyol may be a sugar alcohol. In some embodiments, the stabilizing agent may be the same as the binding agent. In other embodiments, the stabilizing agent may be a different agent.

In some embodiments, the reconstituted cannabis material may further comprise any other suitable excipient. Additional examples of excipients are disclosed in PCT Application No. PCT/CA2020/051460, incorporated by reference above.

In some embodiments, the reconstituted cannabis material may be in the form of a reconstituted cannabis sheet. As used herein, a “sheet” may refer to a relatively thin and relatively flat article. In some embodiments, the reconstituted cannabis material may be in the form of a plurality of strips of a reconstituted cannabis sheet. As used herein, “strip” may refer to a relatively thin and relatively flat piece of material having a length greater than its width. In some embodiments, each strip may be between about 0.5 mm and about 3 mm in width. As one example, each strip may be approximately 1 mm in width.

In other embodiments, the cannabis-based material of the inner core 102 may comprise original (non-reconstituted) cannabis plant matter. In some embodiments, the cannabis-based material may comprise dried cannabis plant matter. The cannabis plant matter may be dried using any suitable drying method, including but not limited to, air-drying or drying in a drying tumbler. In some embodiments, the dried cannabis plant matter may comprise floral matter. In some embodiments, the floral matter may comprise at least one of flowers, flower buds, trichomes, or combinations thereof. In some embodiments, the dried cannabis plant matter may be pressed. The cannabis plant matter may be pressed using any suitable method including, for example, hot pressing. In other embodiments, the dried cannabis matter may be milled. The cannabis plant matter may be milled using any suitable method including, for example, hammer milling.

In some embodiments, the inner core 102 can comprise any combination of the cannabis-based materials described herein. In other embodiments, the inner core 102 may comprise any other suitable type of cannabis-based material.

In some embodiments, the cannabis-based material of the inner core 102 may comprise one or more additives. As used herein, the term “additive” may refer to an additional ingredient that may have biological activity in humans. In some embodiments, the additive may be a substance derived from a cannabis plant. In other embodiments, the additive may be a substance derived from a tobacco plant or any other suitable type of plant. In some embodiments, the additive may be in the form of an extract, an isolate, a distillate, or combinations thereof. In other embodiments, the additive may be in any other suitable form.

In some embodiments, the additive may comprise at least one cannabinoid. Non-limiting examples of suitable cannabinoids include tetrahydrocannabinol (THC), and cannabidiol (CBD). As used herein, “THC” may refer to at least one of Δ⁹-tetrahydrocannabinol, Δ⁹-tetrahydrocannabinolic acid, and isomers thereof. As used herein, “CBD” may refer to at least one of cannabidiol, cannabidiolic acid, and isomers thereof.

In some embodiments, the additive may comprise at least one terpene. Non-limiting examples of suitable terpenes include caryophyllene, beta-caryophyllene (BCP), myrcene, terpinolene, α-pinene, humulene, linalool, and limonene.

In some embodiments, the additive may comprise at least one flavonoid. Non-limiting examples of suitable flavonoids include apigenin, luteolin, quercetin, kaempferol, cannflavin A, cannflavin B, β-sitosterol, vitexin, isovitexin, and orientin.

In some embodiments, the additive may comprise any suitable combination of cannabinoids, terpenes, and/or flavonoids. In other embodiments, the additive may comprise any other suitable additive.

In some embodiments, the additive may be infused into the cannabis-based material. As used herein, “infused” may refer to permeating, impregnating or otherwise integrating the additive into the cannabis-based material. In other embodiments, the additive may be introduced into the cannabis-based material by any other suitable means.

The cannabis-based material may therefore have a desired chemical composition. As used herein, “chemical composition” may refer to the types and concentrations of cannabinoids, terpenes, flavonoids, and/or any other desired chemical compounds. The chemical composition of the cannabis-based material may be based on the chemical compounds naturally present in the cannabis plant matter from which the cannabis-based material was derived in combination with any additives introduced into the cannabis-based material.

Referring again to FIG. 1, the outer sheath 106 may receive the inner core 102 therein. The outer sheath 106 may function to help maintain the shape of the heat-not-burn article 100 and/or to prevent loss of the cannabis-based material forming the rod 104 of the inner core 102. In some embodiments, the outer sheath 106 may be relatively flexible and/or malleable. In other embodiments, the outer sheath 106 may be relatively rigid.

The outer sheath 106 may comprise a sheet 108 of plant-based material. As used herein, “plant-based” may refer to a material that is substantially obtained or derived from plant matter but that may also comprise one or more non-plant ingredients. It will be understood that the thickness of the sheet 108 in FIG. 1 is exaggerated for illustrative purposes and the sheet 108 may be substantially thinner in some embodiments.

In some embodiments, the plant-based material of the sheet 108 may comprise cannabis-based material. In some embodiments, the cannabis-based material may comprise reconstituted cannabis material. In some embodiments, the sheet 108 may comprise the same reconstituted cannabis material as the rod 104. For example, the sheet 108 may comprise a reconstituted cannabis sheet and the rod 104 may comprise a plurality of strips of the same reconstituted cannabis sheet. In other embodiments, the sheet 108 and the rod 104 may comprise different reconstituted cannabis materials.

In some embodiments, the cannabis-based material of the sheet 108 may have a desired chemical composition. The heat-not-burn article 100 may therefore have a desired total chemical composition based on the chemical composition of the cannabis-based material of the rod 104 and that of the sheet 108. In some embodiments, the total chemical composition of the heat-not-burn article 100 may be selected based on a desired potency, aroma, and/or flavor profile. Non-limiting examples of specific chemical compositions are provided in the Examples below.

In other embodiments, the plant-based material of the sheet 108 may comprise a cellulosic material. As used herein, “cellulosic material” may refer to any material comprising cellulose as a significant component. The cellulosic material may be in the form of a paper, a film, or a weaved cellulose material. In some embodiments, the cellulosic material may comprise hemp cellulose. As used herein, “hemp cellulose” may refer to cellulose derived from one or more hemp plants. In some embodiments, the cellulosic material may comprise a mixture of hemp cellulose and cellulose from another type of plant. In other embodiments, the cellulosic material may solely comprise cellulose from one or more other types of plant.

As one specific example, the cellulosic material may comprise a mixture of hemp cellulose, glycerin and water in the form of a film similar to cellophane.

In other embodiments, the sheet 108 may comprise any other suitable type of plant-based material.

In some embodiments, the heat-not-burn article 100 may comprise at least about 90% total cannabis content. As used herein, “total cannabis content” may refer to a total percentage of substances obtained or derived from cannabis plant matter including the cannabis-based material of the inner core 102, optional cannabis-based material of the outer sheath 106 and optional added cannabinoids, terpenes, and/or flavonoids.

In some embodiments, the sheet 108 may be formed into a tubular body 107. The tubular body 107 may receive the rod 104 of the inner core 102 therein. In the embodiment shown in FIG. 1, the tubular body 107 may have approximately the same length as the rod 104. In this embodiment, when the rod 104 is received into the tubular body 107, the opposed ends 111, 113 of the rod 104 may be exposed.

In this embodiment, the tubular body 107 may comprise a seam 109 formed by overlapping opposed longitudinal edges of the sheet 108 and sealing the overlapped edges together. The seam 109 may thereby help hold the sheet 108 in shape of the tubular body 107 to facilitate insertion of the inner core 102 into the tubular body 107.

In some embodiments, the seam 109 is a glued seam in which the opposed longitudinal edges of the sheet 108 are glued together using a suitable adhesive. In other embodiments, the seam 109 may be a glueless seam such as the glueless seams as described in International PCT Application No. PCT/CA2020/051248, incorporated by reference herein. In other embodiments, the seam may be any other suitable type of seam.

In other embodiments, the inner core 102 may be substantially encased in the outer sheath 106. As used herein, “encased” or “encasing” may refer to the substantially covering the inner core 102 such that the inner core 102 is substantially sealed from the outside environment. In some embodiments, the inner core 102 may be encased in the outer sheath 106 by wrapping the sheet 108 around the inner core 102 and sealing opposed ends of the sheet 108. In other embodiments, the sheet 108 may be formed into a receptacle (not shown) and the inner core 102 may be sealed within the receptacle.

In some embodiments, the outer sheath 106 may be secured to the inner core 102. In some embodiments, the outer sheath 106 may be secured to the inner core 102 by securing the tubular body 107 to the rod 104. In some embodiments, the tubular body 107 may be glued to the rod 104 by a suitable adhesive. In other embodiments, the outer sheath 106 may be secured to the inner core 102 by any other suitable securing means.

In some embodiments, the outer sheath 106 may comprise a label (not shown). In some embodiments, the label may be stamped onto the outer sheath 106. In some embodiments, the label may be comprised of non-toxic ink. The label may contain any suitable information relevant to a user. For example, the label may contain: information on the strain (i.e. cultivar) of the cannabis plant from which the cannabis-based material originated; the total chemical composition of the heat-not-burn article 100 such as the types and concentration of cannabinoids, terpenes, and/or flavonoids; a description of the flavor profile; and/or any other relevant information.

In use, the heat-not-burn article 100 may be heated in a heat-not-burn device to a suitable temperature to release volatile components of the cannabis-based material. In some embodiments, the heat-not-burn article 100 may be heated to a temperature less than about 451° F. (approximately 233° C.), which is the temperature at which cannabis typically combusts. For example, the heat-not-burn article 100 may be heated to a temperature between about 200° F. (approximately 93° C.) and about 450° F. (approximately 232° C.). The heat-not-burn article 100 may therefore be heated without combustion to generate an inhalable aerosol and/or vapor, thereby reducing or eliminating undesirable byproducts typically associated with smoke.

Additional examples of heat-not-burn articles will be discussed with reference to FIGS. 2A to 2C.

Another example heat-not-burn article 200 is shown in FIG. 2A. In some embodiments, the heat-not-burn article 200 may have a length L₂ of approximately 10 mm. In other embodiments, the heat-not-burn article 200 may have any other suitable length.

The heat-not-burn article 200 may comprise an inner core 202 and an outer sheath 206. The inner core 202 and the outer sheath 206 may be made of any of the materials described above for the inner core 102 and the outer sheath 106 of the heat-not-burn article 100. The inner core 202 may comprise a compressed rod 204 and the outer sheath 206 may comprise a sheet 208 formed into a tubular body 207.

The heat-not-burn article 200 may have a first end 201 and a second end 203. The rod 204 may have a first end 211 and a second end 213. The first end 211 of the rod 204 may be at the first end 201 of the heat-not-burn article 200 and the second end 213 of the rod 204 may be at the second end of the heat-not-burn article 200.

In some embodiments, the first end 211 of the rod 204 may define a recess 205. The recess 205 may extend longitudinally from the first end 211 into the interior of the rod 204. In some embodiments, the recess 205 may be dimensioned to receive a heating element of a heat-not-burn device (not shown) therein. As one example, the recess 205 may be dimensioned to receive the heating element of an IQOS™ device. Therefore, in some embodiments, when the heat-not-burn article 200 is inserted into the heat-not-burn device, the heating element may be received into the recess 205 to secure the heat-not-burn article 200 within the heat-not-burn device and facilitate heating of the rod 204.

In some embodiments, the second end 213 of the rod 204 may define a second recess (not shown). Alternatively, the rod 204 may define a longitudinal slot (not shown) that extends all the way through the rod 204 from the first end 211 to the second end 213. Therefore, in some embodiments, the heat-not-burn article 200 may be reversible such that the heat-not-burn article 200 may be inserted into the heat-not-burn device with either the first end 201 or the second end 203 facing inward towards the heating element.

Another example heat-not-burn article 210 is shown in FIG. 2B. The heat-not-burn article 210 may comprise an inner core 212 and an outer sheath 216. The inner core 212 may comprise a rod 214 and the outer sheath 216 may comprise a sheet 218 formed into a tubular body 217. The inner core 212 and the outer sheath 216 may be made of any of the materials described above for the inner core 102 and the outer sheath 106 of the heat-not-burn article 100.

In this embodiment, a length L₃ of the heat-not-burn article 210 may be optimized to provide more purchase area to facilitate removal of the heat-not-burn article 210 from a heat-not-burn device (not shown). In some embodiments, the length L₃ of the heat-not-burn article 210 may be approximately 15 mm. In other embodiments, the length L₃ may be any other suitable length.

Another example heat-not-burn article 220 is shown in FIG. 2C, comprising an inner core 222 and an outer sheath 226. In this embodiment, the inner core 222 may comprise a first compressed rod 224 and a second compressed rod 225. The first rod 224 may comprise a first cannabis-based material and the second rod 225 may comprise a second cannabis-based material. In some embodiments, the first and second cannabis-based materials may be the same. In other embodiments, the first and second cannabis-based materials may be different.

The outer sheath 226 may comprise a sheet 228 of plant-based material. The sheet 228 may be formed into a tubular body 227. In FIG. 2C, the sheet 228 is shown as transparent for illustrative purposes to view the first and second rods 224 and 225; however, it will be understood that the sheet 228 may be made of any suitable plant-based material and may be opaque in some embodiments.

The first rod 224 may have a length L₄ and the second rod 225 may have a length L₅. In some embodiments, the length L₄ and the length L₅ may be approximately the same. In other embodiments, the length L₄ and the length L₅ may be different. In some embodiments, lengths L₄ and L₅ may each be approximately 10 mm, resulting in an overall length L₆ of the heat-not-burn article 220 of approximately 20 mm. In other embodiments, the lengths L₄ and L₅, and the overall length L₆ of the heat-not-burn article 220, may be any other suitable lengths.

In some embodiments, the first rod 224 and the second rod 225 may be approximately axially aligned in an end-to-end formation. The first rod 224 may have a first end 221 and a second end 223 and the second rod 225 may have a first end 229 and a second end 231. In this embodiment, a small space 233 may be provided between the second end 223 of the first rod 224 and the first end 229 of the second rod 225. In other embodiments, the second end 223 of the first rod 224 may abut the first end 229 of the second rod 225.

In some embodiments, the first and second rods 224 and 225 are separable. For example, the user may cut or tear the sheet 228 at the space 233 (or at the abutted ends 223, 229 of the first and second rods 224 and 225) to separate the first and second rods 224 and 225. Each separated rod 224 and 225 may have a respective portion of the sheet 228 still wrapped therearound. In some embodiments, the sheet 228 may be weakened at the location of the space 233 (or at the abutted ends 223, 229 of the first and second rods 224 and 225) to facilitate separation of the first and second rods 224 and 225. For example, the sheet 228 may be perforated to facilitate separation of the first and second rods 224 and 225. Therefore, in some embodiments, the heat-not-burn article 220 may be shared by more than one user such that a first user may use the first rod 224 and a second user may use the second rod 225. Alternatively, a single user may use the first rod 224 and save the second rod 225 for future use or vice versa.

The heat-not-burn article 220 may have a first end 230 and a second end 232. In this embodiment, the first end 221 of the first rod 224 is at the first end 230 of the heat-not-burn article 220 and the second end 231 of the second rod 225 is at the second end 232. In some embodiments, a first recess (not shown) may be defined by the first end 221 of the first rod 224 and a second recess (not shown) may be defined at the second end 231 of the second rod 225. The first and second recesses may be similar to the recess 205 of the heat-not-burn article 200 of FIG. 2A. Therefore, in some embodiments, the heat-not-burn article 220 may be reversible such that either the first end 230 or the second end 232 may be inserted into a heat-not-burn device (not shown).

In some embodiments, the first cannabis-based material of the first rod 224 has a first chemical composition and the second cannabis-based material of the second rod 225 has a second chemical composition. The first chemical composition may be the same as or different from the second chemical composition. Therefore, in some embodiments, the heat-not-burn article 220 may provide two rods 224, 225 of different potencies, aromas, and/or flavors that may be used by different users or used by the same user at different times as desired.

In alternative embodiments, the second rod 225 may be omitted and the inner core 222 may comprise the first rod 224 and a filter (not shown). The filter may act as a mouthpiece for the heat-not-burn article 220 and may prevent cannabis-based material of the first rod 224 from entering the user's mouth. In some embodiments, the filter may be approximately the same size and shape as the first rod 224. In other embodiments, the filter may be any other suitable size and shape. The first rod 224 and the filter may be approximately axially aligned in an end-to-end formation in a manner similar to the first rod 224 and the second rod 225 as shown in FIG. 2C.

In some embodiments, the filter may comprise a rolled and/or folded piece of paper, cardboard, or any other suitable material (known as a “crutch”). In other embodiments, the filter may comprise a synthetic filter. As one example, the synthetic filter may comprise a cellulose acetate filter. In other embodiments, the filter may comprise any other suitable type of filter.

In some embodiments, a recess (not shown) may be defined by the first end 221 of the rod 224. The recess may thereby help to orientate the user such that the user inserts the heat-not-burn article 220 into a heat-not-burn device (not shown) in the proper orientation i.e. with the rod 224 facing inward towards the heating element and the filter facing outward towards the user.

Other variations are also possible. In some embodiments, the heat-not-burn article 220 may comprise additional rods such as, for example, a third, fourth, or fifth rod. In some embodiments, the heat-not-burn article 220 may comprise two or more rods in combination with a filter at one end of the heat-not-burn article 220.

A method for making a heat-not-burn article is also provided. The method may be used to make embodiments of the heat-not-burn articles 100, 200, 210 and 220 described above.

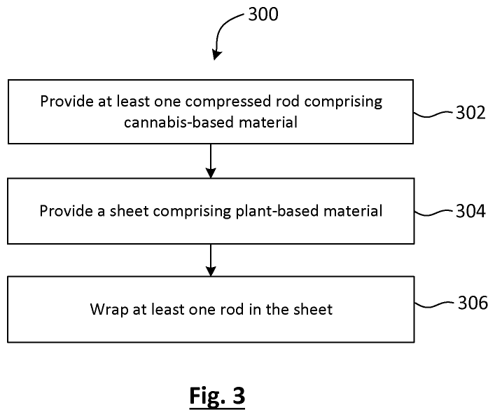

FIG. 3 is a flowchart of an example method 300 for making a heat-not-burn article, according to some embodiments.

At block 302, at least one compressed rod comprising cannabis-based material may be provided. The term “providing” in this context may refer to making, manufacturing, acquiring, buying, or otherwise obtaining the rod.

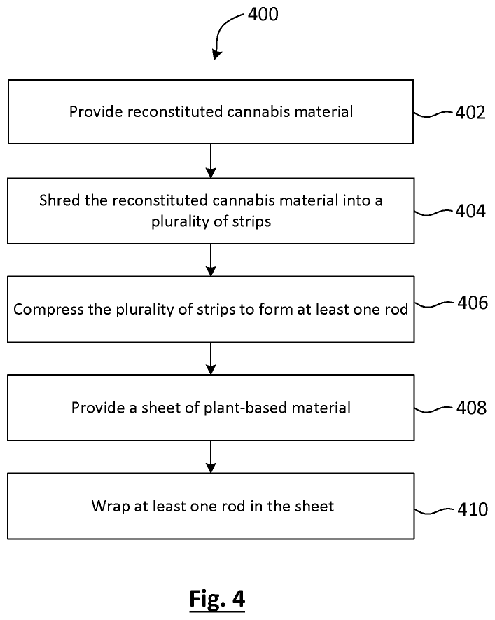

In some embodiments, providing at least one rod may comprise forming the rod(s) from the cannabis-based material. In some embodiments, the cannabis-based material may comprise reconstituted cannabis material. In other embodiments, the cannabis-based material may comprise any other suitable cannabis-based material. In some embodiments, the rod(s) may be formed first and then compressed. In other embodiments, the cannabis-based material may be compressed to form the rod(s). An example of how one or more rods may be provided will be discussed in more detail below with reference to FIG. 4.

In some embodiments, providing at least one rod may comprise providing a first rod and a second rod. In other embodiments, providing at least one rod may comprise providing any suitable number of rods.

In some embodiments, the rod(s) may be selected based on the chemical composition of the cannabis-based material and the desired potency, aroma, and/or flavor of the resulting heat-not-burn article in combination with the sheet. In some embodiments, a first rod may be selected having a first chemical composition and a second rod may be selected having a second chemical composition. The first chemical composition may be the same or different from the second chemical composition.

At block 304, a sheet comprising plant-based material may be provided. The term “providing” in this context may refer to making, manufacturing, acquiring, buying, or otherwise obtaining the sheet.

In some embodiments, providing the sheet may comprise providing a sheet of cannabis-based material such as, for example, a reconstituted cannabis sheet. In some embodiments, the reconstituted cannabis sheet provided at block 304 may be the same reconstituted cannabis material as the rod provided at block 302. In other embodiments, the reconstituted cannabis materials of the rod and the sheet may be different.

In other embodiments, providing the sheet may comprise providing a sheet of cellulosic material. The cellulosic material may comprise any of the cellulosic materials described above. In other embodiments, the sheet may comprise any other suitable type of plant-based material.

In some embodiments, the sheet may be selected based on the chemical composition of the plant-based material and the desired potency, aroma, and/or flavor of the resulting heat-not-burn article in combination with the rod.

At block 306, at least one rod may be wrapped in the sheet. In embodiments in which two or more rods were provided at block 302, the rods may be assembled prior to wrapping the rods in the sheet. In some embodiments, assembling the rods may comprise axially aligning the rods in an end-to-end formation.

In some embodiments, the sheet may be formed into a tubular body around the rod(s). In other embodiments, the sheet may be formed into a tubular body first and then the rod(s) may be inserted into the tubular body. In some embodiments, forming the tubular body may comprise overlapping opposed longitudinal edges of the sheet and sealing the opposed longitudinal edges into a seam. The opposed longitudinal edges may be glued to form a glued seam or, alternatively, may be gluelessly sealed to form a glueless seam. In other embodiments, the tubular body may be sealed by any other suitable means.

In other embodiments, wrapping the rod(s) in the sheet may comprise encasing the rod(s) in the sheet such that the rod(s) are substantially sealed from the outside environment. In some embodiments, encasing the rod(s) in the sheet may comprise wrapping the rod(s) in the sheet and sealing both ends of the sheet. In other embodiments, encasing the rod(s) in the sheet may comprise forming the sheet into a receptacle, inserting the rod(s) into the receptacle, and then sealing the receptacle closed.

In some embodiments, the method 300 may further comprise securing the sheet to the rod(s). The sheet may be secured to the rod(s) by an adhesive or any other suitable means.

In some embodiments, the method 300 may further comprise providing a filter. In some embodiments, the filter may then be assembled with at least one rod prior to the wrapping step at block 306. In some embodiments, the filter and the rod(s) may be approximately axially aligned in an end-to-end formation. In these embodiments, both the rod(s) and the filter may both be wrapped in the sheet at block 306.

In some embodiments, the method 300 may further comprise forming a recess in one or more of the rods. The recess may be configured to receive a heating element of a heat-not-burn device therein. In some embodiments, a respective recess may be formed at each end of a given rod. In other embodiments, a recess may be formed at a first end of the rod, opposite a second end of the rod that is adjacent to a filter or another rod.

In some embodiments, the method 300 may further comprise adding a label to the sheet. In some embodiments, the label may be added by stamping the sheet. In other embodiments, the label may be added by any other suitable means. In some embodiments, the label may be added before the sheet is wrapped around the rod(s) at block 306. In other embodiments, the label may be added after the steps at block 306.

FIG. 4 is a flowchart of another example method 400 for making a heat-not-burn article, showing additional details of how a rod of cannabis-based material may be provided, according to some embodiments. In this example, the cannabis-based material is reconstituted cannabis material.

At block 402, reconstituted cannabis material may be provided. In some embodiments, the reconstituted cannabis material may be provided in the form of a sheet.

In some embodiments, providing the reconstituted cannabis material may further comprise infusing the reconstituted cannabis material with at least one additive. In some embodiments, the additive may be infused during the process for making the reconstituted cannabis material such as by introducing the additive into a slurry of cannabis plant matter and liquid as described in PCT/CA2020/051460, incorporated by reference above. In other embodiments, the additive may be infused into the reconstituted cannabis material after it has been made. For example, the reconstituted cannabis material may be soaked, sprayed, coated, or dripped with the additive. Alternatively, the reconstituted cannabis material may be bathed, rolled, or dipped in the additive.

At block 404, the reconstituted cannabis material may be shredded into a plurality of strips. In some embodiments, the plurality of strips may then be weighed and trimmed to a desired length.

At block 406, the plurality of strips may be compressed into at least one rod. In some embodiments, compressing the plurality of strips may comprise folding and/or pressing the plurality of strips.

In some embodiments, the strips may be compressed to form a “precursor” rod, the precursor rod having a first length. As used herein, “precursor rod” may refer to a rod from which a plurality of individual rods can be produced. The precursor rod may then be cut into at plurality of smaller individual rods having a second length, the second length being shorter than the first length. The second length may be selected based on the desired length of the heat-not-burn article. In other embodiments, the strips may be compressed to form a plurality of individual rods. The length of each individual rod may be selected based on the desired length of the heat-not-burn article.

At block 408, a sheet of plant-based material may be provided. In some embodiments, the sheet may comprise reconstituted cannabis material. In some embodiments, the reconstituted cannabis material may be the same as the reconstituted cannabis material provided at block 402. In other embodiments, the sheet may comprise any other suitable plant-based material. In some embodiments, a larger sheet may be provided to wrap the precursor rod as described above. In other embodiments, a plurality of smaller, individual sheets may be provided, each of the plurality of individual sheets being dimensioned to wrap one or more individual rods.

At block 410, at least one rod may be wrapped in the sheet. In some embodiments, the precursor rod may be wrapped in the sheet. The wrapped precursor rod may then be cut into a plurality of smaller, individual wrapped rods, each of the individual wrapped rods comprising an individual heat-not-burn article. In other embodiments, one or more individual rods may be wrapped in a respective individual sheet to form an individual heat-not-burn article. In other embodiments, each individual sheet may be formed into a receptacle to receive one or more respective individual rods therein. In other embodiments, the sheet may be continuously wrapped around the plurality of individual rods in a flow-wrapping process as described in more detail below. The steps at block 410 may otherwise be similar to the steps at block 306 of the method 300 as described above.

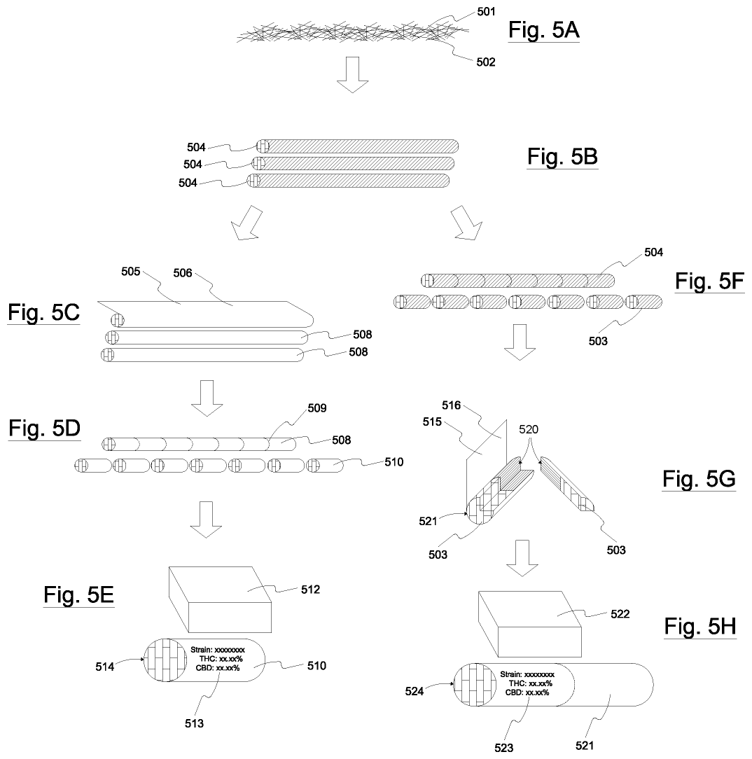

Another example method for making heat-not-burn articles is shown illustratively in FIGS. 5A to 5H. As shown in FIG. 5A, cannabis-based material 501 may be provided. In this example, the cannabis-based material 501 may comprise reconstituted cannabis material. The reconstituted cannabis material may be shredded into a plurality of strips 502. As shown in FIG. 5B, the plurality of strips 502 may be compressed to form at least one precursor rod 504.

FIGS. 5C to 5E show one example of a series of steps for making a heat-not-burn article 514 from the precursor rods 504 of FIG. 5B. As shown in FIG. 5C, each precursor rod 504 may be wrapped in a sheet 506 of plant-based material 505 to form a respective wrapped precursor rod 508. In this embodiment, the plant-based material 505 may comprise reconstituted cannabis material. In some embodiments, the reconstituted cannabis material 505 may be the same as the reconstituted cannabis material 501. In other embodiments, the reconstituted cannabis materials 501 and 505 may be different. Optionally, the sheet 506 may be secured to the precursor rod 504 by an adhesive or any other suitable means.

As shown in FIG. 5D, in some embodiments, each wrapped precursor rod 508 may be marked or perforated by markings 509 to indicate where the wrapped rod 508 should be cut based on a desired length of the heat-not-burn article 514. The wrapped precursor rod 508 may then be cut into a plurality of smaller, individual wrapped rods 510. The individual wrapped rods 510 may all have the same length or, alternatively, the individual wrapped rods 510 may have two or more different lengths.

As shown in FIG. 5E, each individual wrapped rod 510 may be stamped by a stamping device 512 with a label 513. The label 513 may indicate the strain (cultivar) of cannabis used to produce the heat-not-burn article 514, the THC and CBD concentration, and/or any other useful information. The heat-not-burn article 514 may then be ready for use by a user.

FIGS. 5F to 5H show an alternative series of steps for making a heat-not-burn article 524 from the precursor rods 504 of FIG. 5B. As shown in FIG. 5F, each precursor rod 504 may be cut into a plurality of smaller, individual rods 503. The individual rods 503 may all have the same length or, alternatively, the individual rods 503 may have two or more different lengths.

As shown in FIG. 5G, a filter 520 may also be provided. The filter 520 may then be assembled with one of the individual rods 503. In some embodiments, a plurality of filters 520 may be provided and each filter 520 may be assembled with a respective individual rod 503. In some embodiments, each individual rod 503 may be approximately axially aligned with a respective filter 520 in an end-to-end formation. The individual rod 503 and the filter 520 may then be wrapped in an individual sheet 516 of a plant-based material 515 to form a wrapped rod/filter assembly 521. Note that in FIG. 5G, a portion of wrapped rod/filter assembly 521 is shown cut away for illustrative purposes to show the interior of the individual rod 503 and the filter 520. The plant-based material 515 may comprise reconstituted cannabis material or any other suitable plant-based material.

As shown in FIG. 5H, the wrapped rod/filter assembly 521 may be stamped by a stamp 522 with a label 523. The stamp 522 and the label 523 may be similar to the stamping device 512 and the label 513 of FIG. 5E as described above. The heat-not-burn article 524 may then be ready for use by the user.

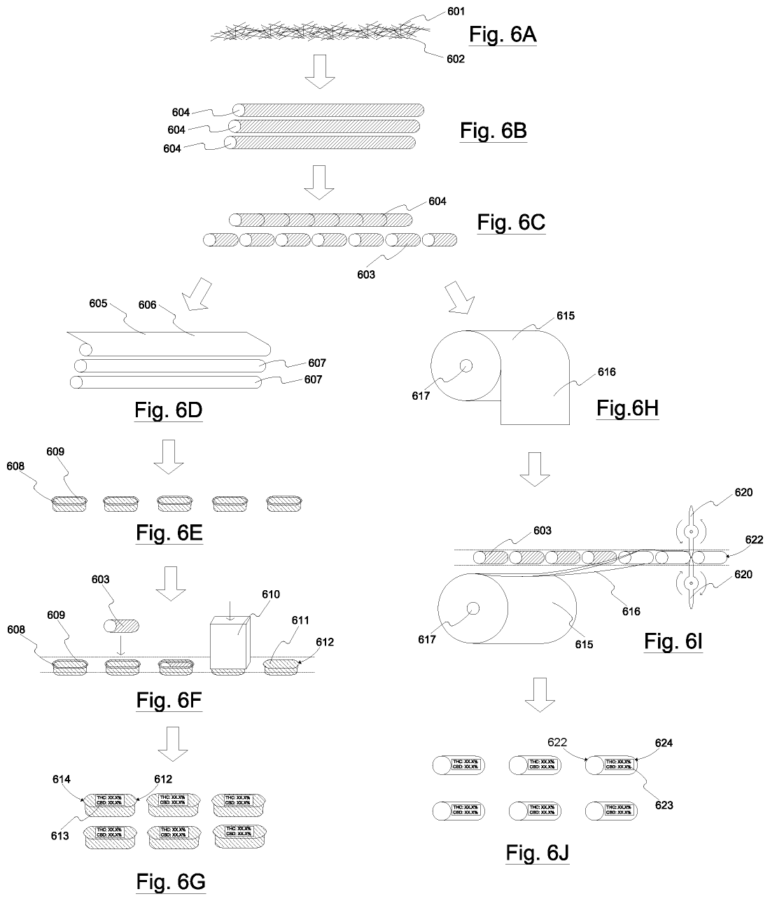

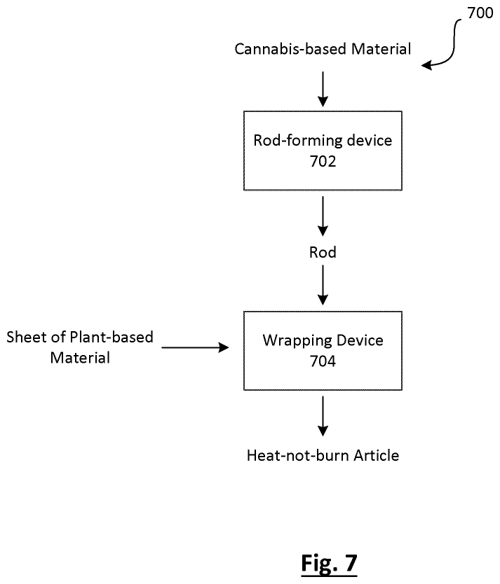

Another example method for making heat-not-burn articles is shown illustratively in FIGS. 6A to 6J. As shown in FIG. 6A, a reconstituted cannabis-based material 601 may be provided and shredded into a plurality of strips 602. As shown in FIG. 6B, the plurality of strips 602 may be compressed to form at least one precursor rod 604. As shown in FIG. 6C, each precursor rod 604 may be cut into a plurality of smaller, individual rods 603.

FIGS. 6D to 6G show one example of a series steps to make a heat-not-burn article 614 from the individual rods 603 of FIG. 6C. As shown in FIG. 6D, a sheet 606 of plant-based material 605 may be cut from a bobbin 607. In this example, the plant-based material 605 may comprise weaved hemp cellulose. As shown in FIG. 6E, the sheet 606 may be cut into a plurality of smaller, individual sheets 608 and each of the individual sheets 608 may be formed into a receptacle 609. Each receptacle 609 may be dimensioned to receive an individual rod 603 therein. In some embodiments, a plurality of closure pieces 611 (visible in FIG. 6F) may also be cut from the sheet 606 to close the receptacle 609 as described below.

As shown in FIG. 6F, each individual rod 603 may be inserted into a respective receptacle 609. A pressing device 610 may be used to press a respective closure piece 611 onto each receptacle 609 to seal the individual rod 603 therein, thereby forming an encased rod 612.

As shown in FIG. 6G, each encased rod 612 may be stamped by a stamping device (not shown) with a label 613. The label 613 may be similar to the label 513 of FIGS. 5E and 5H. The heat-not-burn article 614 may then be ready for use by a user.

FIG. 6H to 6J show an alternative series of steps to form a heat-not-burn article 624 from the individual rods 603 of FIG. 6C using a flow-wrapping technique. As used herein, “flow-wrapping” may refer to wrapping a sheet around a plurality of individual rods while cutting and sealing the sheet at both ends of each individual rod.

As shown in FIG. 6H, a sheet 616 of plant-based material 615 may be provided on a bobbin 617. In this example, the plant-based material 615 may comprise hemp cellulose paper or any other suitable type of plant-based material.

As shown in FIG. 6I, the individual rods 603 may be moved longitudinally with respect to the bobbin 617. In some embodiments, the rods 603 may be moved along a conveyor belt (not shown). In other embodiments, the rods 603 may be moved by any other suitable means. As the individual rods 603 move past the bobbin 617, the sheet 616 may be continuously unspooled from the bobbin 617 and wrapped around the plurality of individual rods 603. As the sheet 616 wraps around each individual rod 603, a sealing mechanism 620 may cut and seal the sheet 616 at both ends of each individual rod 603 to form an encased rod 622. In some embodiments, the sealing mechanism 620 may use at least one of heat and pressure to seal the sheet 616 at both ends of the rod 603. In some embodiments, the application of heat and pressure may simultaneously cut and seal the sheet 616.

As shown in FIG. 6J, each encased rod 622 may be stamped by a stamping device (not shown) with a label 623. The label 623 may be similar to the label 513 of FIGS. 5E and 5H. The heat-not-burn article 624 may then be ready for use by a user.

FIG. 7 shows an example system 700 for making a heat-not-burn article, according to some embodiments. The system 700 may be used to implement embodiments of the methods described herein.

The system 700 may comprise a rod-forming device 702 and a wrapping device 704. The rod-forming device 702 may be configured to form cannabis-based material into a rod. In some embodiments, the rod-forming device 702 may be configured to compress the cannabis-based material to form the rod. In some embodiments, the rod-forming device 702 may be configured to fold and/or press a plurality of strips of reconstituted cannabis material to form the rod.

The wrapping device 704 may be configured to wrap the rod of cannabis-based material in a sheet of plant-based material. In some embodiments, the wrapping device 704 may be configured to form the sheet into a tubular body. In some embodiments, the wrapping device 704 may comprise a sealing mechanism to seal opposed longitudinal edges of the sheet into a seam.

In other embodiments, the wrapping device 704 may be configured to form the sheet into a receptacle and seal the rod in the receptacle. In other embodiments, the wrapping device 704 may comprise a flow-wrapping device. The flow-wrapping device may be configured to continuously wrap the sheet around a plurality of individual rods. In some embodiments, the flow wrapping device may include a conveyor belt or any other suitable means to move the plurality of rods with respect to the sheet. In some embodiments, the flow-wrapping device may include a sealing mechanism to cut and seal the sheet at both ends of each rod. In some embodiments, the sealing mechanism may comprise rolling packet sealers. In other embodiments, the sealing mechanism may comprise any other suitable sealing mechanism.

In some embodiments, the system 700 may further comprise at least one cutting device (not shown). In some embodiments, at least one cutting device may comprise a shredding device to shred the cannabis-based material into the plurality of strips used to form the rod. In some embodiments, at least one cutting device may comprise a rod-cutting device to cut the rod formed by the rod-forming device 702 and/or a wrapped rod formed by the wrapping device 704. The rod-cutting device may be configured to cut the rod and/or wrapped rod to a desired length based on the desired length of heat-not-burn article.

In some embodiments, the system 700 may further comprise an assembly device (not shown). In some embodiments, the assembly device comprises an assembly drum. The assembly device may be configured to assemble two or more rods of cannabis-based material or one or more rods and a filter. In some embodiments, the assembly device may be configured to approximately axially align the rod(s) and the filter in end-to-end formation.

In some embodiments, the system 700 may further comprise a stamping device (not shown). The stamping device may be configured to stamp a suitable label onto the heat-not-burn article.

### EXAMPLES

Without any limitation to the foregoing, specific examples of heat-not-burn articles will be described below.

### Example 1—Natural Cannabis Citrus Flavored Heat-Not-Burn Articles

Heat-not-burn articles with natural citrus flavoring may be made from reconstituted cannabis material. Briefly, a sheet of reconstituted cannabis material may be provided comprising 10 mg THC per square inch, 100 mg CBD per square inch, and 2 mg limonene per square inch. The sheet may be shredded into strips and compressed into a precursor rod. The precursor rod may be wrapped in another sheet of the same reconstituted cannabis material. The wrapped precursor rod (referred to as a “cylinder” in Table 1 below) may comprise a total of approximately 10 square inches of reconstituted cannabis material. The wrapped precursor rod may be cut into 4 individual wrapped rods (i.e. 4 individual heat-not-burn articles) and packaged accordingly.

The chemical composition of the reconstituted cannabis material and resulting end products is summarized in Table 1 below. Each heat-not-burn article (“unit”) contains 25 mg THC, 250 mg CBD, and 5 mg limonene. Limonene is a naturally occurring terpene that may impart a lemon flavor and aroma to the heat-not-burn articles.

TABLE 1

| Additive | Amount | Measurement | Total/cylinder | Total/unit |
|---|---|---|---|---|
| THC | 10 | mg/sq. inch | 100 | 25 |
| CBD | 100 | mg/sq. inch | 1000 | 250 |
| Limonene | 2 | mg/sq. inch | 20 | 5 |
| Cylinder raw size | 10 | sq. inch per | | |
| | | cylinder | | |

### Example 2—Natural Cannabis Pepper Flavored Heat-Not-Burn Articles

Heat-not-burn articles with natural pepper flavor may also be made from reconstituted cannabis material. The heat-not-burn articles may be made in a similar manner as described in Example 1 above. Tables 2, 3, 4, and 5 below show several possible chemical compositions of reconstituted cannabis materials and resulting end products. These compositions include beta-caryophyllene (BCP), a naturally occurring terpene that imparts a black pepper flavor to the heat-not-burn articles.

TABLE 2

| Additive | Amount | Measurement | Total/cylinder | Total/unit |
|---|---|---|---|---|
| THC | 10 | mg/sq. inch | 100 | 25 |
| CBD | 100 | mg/sq. inch | 1000 | 250 |
| BCP | 0.2 | mg/sq. inch | 2 | 0.5 |
| Cylinder raw size | 10 | sq. inch per | | |
| | | cylinder | | |

TABLE 3

| Additive | Amount | Measurement | Total/cylinder | Total/unit |
|---|---|---|---|---|
| THC | 4 | mg/sq. inch | 40 | 10 |
| CBD | 4 | mg/sq. inch | 40 | 10 |
| BCP | 0.1 | mg/sq. inch | 1 | 0.25 |
| Cylinder raw size | 10 | sq. inch per | | |
| | | cylinder | | |

TABLE 4

| Additive | Amount | Measurement | Total/cylinder | Total/unit |
|---|---|---|---|---|
| THC | 10 | mg/sq. inch | 100 | 25 |
| CBD | 100 | mg/sq. inch | 1000 | 250 |
| BCP | 2 | mg/sq. inch | 20 | 5 |
| Cylinder raw size | 10 | sq. inch per | | |
| | | cylinder | | |

TABLE 5

| Additive | Amount | Measurement | Total/cylinder | Total/unit |
|---|---|---|---|---|
| THC | 0 | mg/sq. inch | 0 | 0 |
| CBD | 10 | mg/sq. inch | 100 | 25 |
| BCP | 0.1 | mg/sq. inch | 1 | 0.25 |
| Cylinder raw size | 10 | sq. inch per | | |
| | | cylinder | | |

Beta-caryophyllene is the primary sesquiterpene contributing to the spiciness of black pepper (Hartsel et al. “Cannabis sativa and Hemp.” Nutracueticals, edited by Ramesh C. Gupta, Academic Press, 2016, pp. 735-754; Gertsch et al. “Beta-caryophyllene is a dietary cannabinoid.” Proc Natl Acad Sci USA. 2008 Jul. 1; 105(26):9099-104). Studies have shown that inhaling vapors from black pepper essential oils can suppress certain smoking withdrawal symptoms (Rose et al., “Inhalation of vapor from black pepper extract reduces smoking withdrawal symptoms.” Drug Alcohol Depend. 1994 February; 34(3):225-9; Cordell et al. “The effects of aromatherapy on nicotine craving on a U.S. campus: a small comparison study.” J Altern Complement Med. 2013 August; 19(8):709-13). Therefore, the heat-not-burn articles in this Example may be used to reduce symptoms of withdrawal from nicotine or tobacco products. In particular, heat-not-burn articles made from reconstituted cannabis material comprising about 0.01 mg to about 2 mg of BCP, about 0 to about 100 mg THC, and/or about 0 to about 100 mg CBD per square inch may be useful for this application.

### Example 3—Sleep Aid Heat-not-Burn Articles

Heat-not-burn articles with may also be made from reconstituted cannabis material for use as sleep aids to improve sleep and/or reduce insomnia. The heat-not-burn articles may be made in a similar manner as described in Example 1 above. Table 6 below shows an example of a possible chemical composition of reconstituted cannabis material and resulting end products. This composition includes the terpenes terpinolene, myrcene, and caryophyllene.

TABLE 6

| Additive | Amount | Measurement | Total/cylinder | Total/unit |
|---|---|---|---|---|
| THC | 4 | mg/sq. inch | 40 | 10 |
| CBD | 4 | mg/sq. inch | 40 | 10 |
| Terpinolene | 0.1 | mg/sq. inch | 1 | 0.25 |
| Myrcene | 0.4 | mg/sq. inch | 4 | 1 |
| Caryophyllene | 0.2 | mg/sq. inch | 2 | 0.5 |
| Cylinder raw size | 10 | sq. inch per | | |
| | | cylinder | | |

Various modifications besides those already described are possible without departing from the concepts disclosed herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although particular embodiments have been shown and described, it will be appreciated by those skilled in the art that various changes and modifications might be made without departing from the scope of the disclosure. The terms and expressions used in the preceding specification have been used herein as terms of description and not of limitation, and there is no intention in the use of such terms and expressions of excluding equivalents of the features shown and described or portions thereof.

It is to be understood that a combination of more than one of the approaches described above may be implemented. Embodiments are not limited to any particular one or more of the approaches, methods or apparatuses disclosed herein. One skilled in the art will appreciate that variations, alterations of the embodiments described herein may be made in various implementations without departing from the scope of the claims.

1. A heat-not-burn article comprising:
an inner core comprising at least one compressed rod of cannabis-based material; and
an outer sheath comprising a sheet of plant-based material, the outer sheath receiving the inner core therein.

2. The heat-not-burn article of claim 1, wherein the cannabis-based material comprises reconstituted cannabis material.

3. The heat-not-burn article of claim 2, wherein the reconstituted cannabis material is in the form of a plurality of strips of a reconstituted cannabis sheet.

4. The heat-not-burn article of claim 1, wherein the cannabis-based material comprises dried cannabis plant matter.

5. The heat-not-burn article of claim 1, wherein the sheet of plant-based material comprises reconstituted cannabis material.

6. The heat-not-burn article of claim 1, wherein the sheet of plant-based material comprises cellulosic material.

7. The heat-not-burn article of claim 1, wherein the inner core is substantially encased in the outer sheath.

8. The heat-not-burn article of claim 7, wherein the sheet of plant-based material is formed into a receptacle and the at least one rod is sealed within the receptacle.

9. The heat-not-burn article of claim 1, wherein the cannabis-based material comprises at least one additive.

10. The heat-not-burn article of claim 9, wherein the at least one additive comprises at least one of a cannabinoid, a terpene, and a flavonoid.

11. The heat-not-burn article of claim 10, wherein the terpene comprises limonene or beta-caryophyllene.

12. The heat-not-burn article of claim 1, wherein the heat-not-burn article has a total cannabis content of at least about 90%.

13. The heat-not-burn article of claim 1, wherein the at least one rod comprises a first rod and a second rod, the first rod and the second rod being approximately axially aligned in an end-to-end formation.

14. The heat-not-burn article of claim 13, wherein the first rod comprises a first chemical composition and the second rod comprises a second chemical composition, the first chemical composition being different from the second chemical composition.

15. The heat-not-burn article of claim 1, wherein the inner core further comprises a filter, the filter and the at least one rod being approximately axially aligned in an end-to-end formation.

16. The heat-not-burn article of claim 1, wherein each rod of the at least one rod has a first end and a second end and wherein at least one of the first end and the second end defines a recess to receive a heating element of a heat-not-burn device therein.

17. A method for making a heat-not-burn article, the method comprising:
providing at least one compressed rod comprising cannabis-based material;
providing a sheet comprising plant-based material; and
wrapping the at least one rod in the sheet.

18. The method of claim 17, wherein providing the at least one rod comprises:
providing reconstituted cannabis material;
shredding the reconstituted cannabis material into a plurality of strips; and
compressing the plurality of strips to form the at least one rod.

19. The method of claim 18, wherein providing the reconstituted cannabis material further comprises infusing the reconstituted cannabis material with at least one additive.

20. The method of claim 17, wherein wrapping the at least one rod in the sheet comprises substantially encasing the at least one rod in the sheet.

21-23. (canceled)
